(19) United States
(12) Patent Application Publication
(10) Pub. No.: US 2016/0109563 A1
(43) Pub. Date: Apr. 21, 2016

(54) BEAMFORMING APPARATUS, ULTRASOUND IMAGING APPARATUS, AND BEAMFORMING METHOD

(71) Applicant: ALPINION MEDICAL SYSTEMS CO., LTD., Seoul (KR)

(72) Inventor: Moo Ho BAE, Seoul (KR)

(73) Assignee: ALPINION MEDICAL SYSTEMS CO., LTD., Seoul (KR)

(21) Appl. No.: 14/918,016

(22) Filed: Oct. 20, 2015

(30) Foreign Application Priority Data
Oct. 21, 2014 (KR) 10-2014-0142895

(51) Int. Cl. G01S 7/52; A61B 8/14; A61B 8/08

(52) U.S. Cl. CPC G01S 7/52085; A61B 8/5207; A61B 8/14

(57) ABSTRACT

A beamforming apparatus, an ultrasound imaging apparatus, and a beamforming method are disclosed. The beamforming apparatus according to an exemplary embodiment may include a filter to select predetermined first columns, which correspond to low-frequency components, among columns that composes a transform function; and a beamforming processor to transform an input signal to another space by using a transform function composed of the predetermined selected first columns, and generate a beam signal through signal processing in the transformed space.

### CROSS-REFERENCE TO RELATED APPLICATION(S)

This application claims the benefit under 35 U.S.C. §119(a) of Korean Patent Application No. 10-2014-0142895, filed on Oct. 21, 2014, in the Korean Intellectual Property Office, the entire disclosure of which is incorporated herein by reference for all purposes.

### BACKGROUND

1. Field

The following description relates to a beamforming technology.

2. Description of the Related Art

An ultrasound imaging apparatus acquires images related to a tomographic image, a bloodstream, etc. regarding objects, e.g., all types of tissues or structures within a human's body. Such an ultrasound imaging apparatus is relatively small and low-priced; is capable of displaying real-time images; and because there is no danger of radiation exposure to x-rays, etc., is widely used in medical fields, e.g., hospitals dealing with heart, abdomen, and urinary system diseases, and obstetrics and gynecology.

The ultrasound imaging apparatus irradiates ultrasonic waves towards a target area within the objects, collects echo ultrasonic waves that have been reflected from the target area, and generates the ultrasound image based on the information on the collected ultrasonic waves. To that end, the ultrasound imaging apparatus performs beamforming to estimate a size of a reflected wave in specific space with regard to a plurality of channel data coming from the echo signals that have been collected by the ultrasonic probe. The beamforming is to correct the time differences of the ultrasonic signals that have been input through a plurality of ultrasonic sensors, e.g., transducers; emphasize the signal of a specific position by adding a predetermined weight value to each input ultrasonic signal, i.e., a beamforming coefficient; or focus the ultrasonic signals by relatively decreasing the signals of other positions. By the beamforming, the ultrasound imaging apparatus may generate appropriate ultrasound images for identifying the inner structure of the object, which are then displayed to a user.

### SUMMARY

Provided are a beamforming apparatus, an ultrasound imaging apparatus, and a beamforming method so as to reduce the computation amount required for beamforming to reduce a resource use amount of the beamforming apparatus required for the beamforming, and additionally improve the computation speed.

In one general aspect, a beamforming apparatus includes: a filter to, among components of a transform function, remove high-frequency components and select low-frequency components; and a beamforming processor to transform an input signal to another space by using the transform function composed of the selected low-frequency components, and generate a beam signal through signal processing in the transformed space.

Here, the transform function may be composed of orthogonal polynomials. The orthogonal polynomials may be one of Hermite polynomials, Laguerre polynomials, Jacobi polynomials, Gegenbauer polynomials, Chebyshev polynomials, the Legendre polynomials.

A transform function V may be Legendre polynomials P, where P=[P0, P1, . . . , PL−1]T,

$${p}_{mk} = {\sum }_{n = 0}^{k} {m}^{n} {c}_{nk} ,$$

Pk is a k-th column of P, and cₙₖ is determined by a Gram-Schmidt orthonormalization process. The beamforming processor may perform beamforming by using a minimum variance that is based on the orthogonal polynomials in the transformed space.

The beamforming processor may include: a transformer to generate a transform signal with regard to an input signal by using the transform function; a weight value calculator to calculate a transform signal weight value, which is a weight value with regard to the transform signal; and a combiner to generate a beam signal by using the transform signal and the transform signal weight value. The weight value calculator may calculate the weight value from a spatial covariance matrix, which is generated through spatial smoothing for generating the spatial covariance matrix from the transform signal.

In another general aspect, an ultrasound imaging apparatus includes: a transducer to irradiate ultrasonic waves to a subject, receive a signal of the ultrasonic waves reflected from the subject, transform the received ultrasonic waves, and output a plurality of the ultrasonic signals; a beamformer to transform, to another space, the signal of the ultrasonic waves, which has been input through the transducer, by using a transform function, generate a beam signal through signal processing in the transformed space, among components of the transform function, remove high-frequency components, and select and process low-frequency components; and an image generator to generate an image by using a beam signal, which has been generated by the beamformer.

In yet another general aspect, a beamforming method includes: removing high-frequency components and selecting low-frequency components among components of a transform function; and transforming an input signal to another space by using the transform function composed of the selected low-frequency components, and generating a beam signal through signal processing in the transformed space.

Other features and aspects may be apparent from the following detailed description, the drawings, and the claims.

### BRIEF DESCRIPTION OF THE DRAWINGS

FIG. 1 is a diagram illustrating an example of a beamforming apparatus according to an exemplary embodiment.

FIG. 2 is a detailed diagram illustrating an example of a beamforming processor according to an exemplary embodiment.

FIG. 3 is a diagram illustrating an example of an ultrasound imaging apparatus according to an exemplary embodiment.

FIGS. 4A and 4B are diagrams illustrating an example of high-frequency component being removed through only spatial smoothing according to an exemplary embodiment.

FIG. 5 is a diagram illustrating an example of comparing the continuous wave (CW) beam patterns of a transform function P based on Legendre Polynomials and the transform function of Fourier transform functions B and principal component analysis-based minimum variance beamforming (‘PCA MV BF’) method, according to an exemplary embodiment.

FIG. 6 is a flowchart illustrating an example of a beamforming method according to an exemplary embodiment.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. Also, descriptions of well-known functions and constructions may be omitted for increased clarity and conciseness.

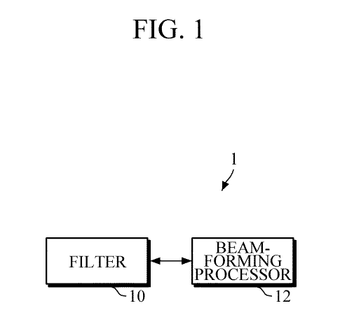

FIG. 1 is a diagram illustrating an example of a beamforming apparatus according to an exemplary embodiment.

Referring to FIG. 1, a beamforming apparatus 1 includes a filter 10 and a beamforming processor 12.

FIG. 1 illustrates the beamforming apparatus 1 including only relevant elements to exemplary embodiments. Thus, it will be understood by those skilled in the art that FIG. 1 may further include other general-use elements not illustrated therein.

Also, at least some elements composing the beamforming apparatus 1 illustrated in FIG. 1 may be one processor or a plurality of processors. The processors may be implemented in a plurality of the arrays of logic gates or in the combination of a general-use microprocessor and a memory storing a program that is executable in such a microprocessor. In addition, it will be understood by those skilled in the art that the processor may be implemented in other types of hardware.

The beamforming apparatus 1 receives an echo signal that has been reflected from a subject and generates a reception beam therefrom. Here, the subject may be, for example, abdomen, heart, etc., of a human body, or the echo signal may be an ultrasonic signal that has been reflected from the subject, to which exemplary embodiments are not, however, limited.

The beamforming processor 12 transforms an input signal of element space into transformed space that is another space, by using a transform function, and generates a beam signal in the transformed space through signal processing to then output the beam signal. The transform function may be expressed as a transform matrix.

The beamforming method of the beamforming processor 12 varies. According to an exemplary embodiment, the beamforming processor 12 uses a beamspace adaptive beamforming method (hereinafter referred to as ‘BABF’), a principal component analysis-based minimum variance beamforming method (hereinafter referred to as ‘PCA MV BF’), etc. The above-mentioned methods have common features of transforming the input signal of the element space into another space by using a transform function that is an orthonormal basis matrix and reducing the dimension of a spatial covariance matrix regarding each input signal, which is input in a plurality of channels, through signal processing, such as the approximation that leaves only essential components in the transformed space. Accordingly, the inverse of the covariance matrix may be very simply calculated.

The beamforming processor 12 uses a transform function that is composed of orthogonal polynomials when transforming the space. The orthogonal polynomials are a series of polynomials that meet the orthogonal relation. The orthogonal polynomials may be, for example, any one of the Hermite polynomials, the Laguerre polynomials, the Jacobi polynomials, the Gegenbauer polynomials, the Chebyshev polynomials, or the Legendre polynomials. Hereinafter, the orthogonal polynomials will be described based on the Legendre polynomials, to which the orthogonal polynomials are not, however, limited.

The beamforming processor 12 uses, as a transform function, the orthonormal matrix that is composed of the Legendre polynomials. Particularly, the beamforming processor 12 may use the Legendre polynomials as a transform function in an MV BF method. In such a case, the beamforming processor 12 may maintain its performance even by drastically reducing the computation amount of the MV BF. Hereinafter, a method of using, as a transform function, the matrix composed of the Legendre polynomials to transform the input signal of the element space into the transformed space is referred to as the Legendre polynomials-based (LP) MV BF method.

The beamforming processor 12 performs the approximation on the MV BF more precisely than BA BF through the LP MV BF method. Furthermore, while properly estimating a spatial covariance matrix by appropriately using the properties of a Fourier transform basis function and the Legendre polynomials in BA BF, the beamforming processor 12 very simply and effectively calculates the spatial smoothing for preventing the signal cancellation that is caused by the coherence of each of the channel signals.

The filter 10 is a low-pass filter, which filter and acquire the low-frequency components in the element space, which corresponds to a side lobe that is close to the front of a beam pattern. Here, the filter 10 selects some of the first columns, which represent the low-frequency component, among the columns composing the transform function. The reason why the low-pass filtering is performable is due to the fact that when the spatial smoothing is performed, the high-frequency components, which exist far away from the front and in which the beam pattern is formed, are mostly removed. Here, the high-frequency and low-frequency components indicate that the frequency components in the lateral direction in terms of space are, respectively, high-frequency and low-frequency.

In a case in which the beamforming processor 12 performs the beamforming in the transformed space by using the MV BF method, the filter 10 according to an exemplary embodiment may drastically reduce the computation amount, which is required for calculating the inverse of a spatial covariance matrix by using only some essential components in the transformed space. For example, the beamforming processor 12 may reduce the dimension of the spatial covariance matrix by acquiring some of the first columns from the transform matrices and transforming their input signals. Some of the first columns from the transform matrices indicate the essential components in calculating the MV BF.

In a case in which the beamforming processor 12 performs the beamforming using a Fourier transform matrix for the BA BF in the transformed space (which is also called as Butler matrix), the filter 10 according to an exemplary embodiment uses some of the first columns among the Fourier transform matrices. Some of the first columns represent the low-frequency components, which correspond to a focal point direction and the beam component that is close to the focal point. Assuming that the interference is mostly generated near the direction of the front, the beamforming processor 12 may effectively reduce the dimension of the spatial covariance matrix by using only such columns.

In a case in which the beamforming processor 12 performs the beamforming by using a PCA MV BF method that applies the PCA to the set of a lot of MV weight values that have been calculated by using the MV BF method, the filter 10 may reduce the dimension of the spatial covariance matrix by using only some of the first columns. Such columns may also present the front direction and the beam component that is near the front.

In a case in which the beamforming processor 12 performs an orthogonal polynomials-based MV BF method, the filter 10 according to an exemplary embodiment uses only some of the first columns of the transform function that is composed of the orthogonal polynomials. Since such columns of the transform function represent the components from low-frequency to high-frequency in order, some of the first columns correspond to the low-frequency component the same as the Fourier transform function.

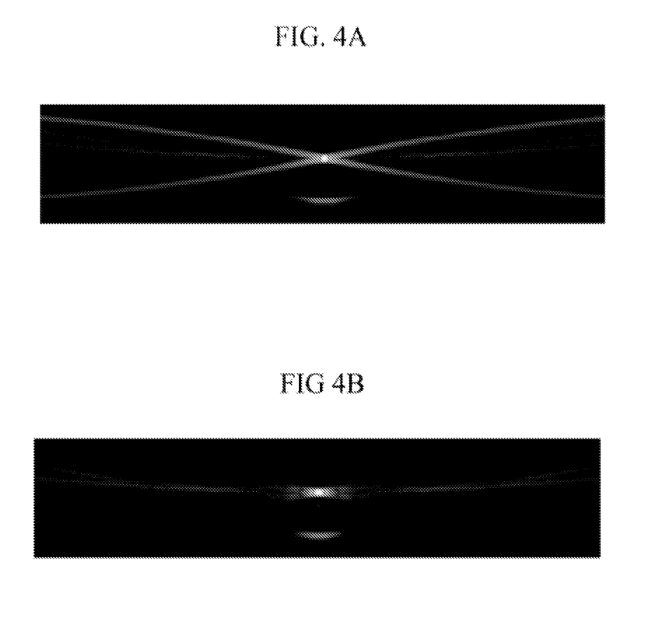

An X-type and long side lobe, which is observed in a delay-and-sum beamforming (hereinafter referred to as ‘DAS BF’) method, may be removed through the spatial smoothing of the MV BF. Although the dimensionality reduction is performed to, by using such properties, deal with only the properties of the side lobe that is near the front and remove other high-frequency properties in the MV BF that uses the Legendre polynomials-based transform function, the properties of the MV BF may be very well maintained. It will be confirmed that the high-frequency properties are removed only through the spatial smoothing, with reference to FIGS. 4A and 4B that will be described later.

Hereinafter, as the method for improving the performance of the beamforming apparatus 1, particularly, the lateral direction resolution and contrast resolution, the background, in which the orthogonal polynomials-based transform function, such as the Legendre polynomials, is used when performing the MV BF, and its function will be specifically described.

An ultrasonic diagnostic apparatus may use a DAS BF method to focus an ultrasonic beam towards the desired direction. In performing the DAS BF, weight values of a type appropriate for a reception signal coming from the array elements are required to be applied thereto so as to lower the level of a clutter that is generated by an echo signal coming from the undesired direction, wherein there is a restriction on the width of a main lobe that is required to be widened.

As a method for improving the performance of an ultrasonic diagnostic apparatus by solving such a restriction, there is a method of applying the MV BF (which is also called Capon beamforming named after the inventor's name). The MV BF may be used to pass the signal, coming from the desired direction, as a unity gain which is 1, and optimally reduce the signal, coming from another direction by calculating and applying an optimum weight value (i.e., apodization function) for each reception focal point based on the input data. Accordingly, the MV BF may be used to simultaneously improve the spatial resolution and the contrast resolution since, while lowering the level of the clutter, reducing the width of the main lobe, which is different from the DAS BF.

One of the biggest disadvantages of the MV BF is that compared to the DAS BF, the required computation amount is too large to be applied to the ultrasonic diagnostic apparatus, which has its importance in the real-time. That is, the MV BF method may need the large amount of the computation because the inverse of the spatial covariance matrix is required to be obtained. Thus, the computation amount is required to be reduced while the performance of the MV BF is not reduced if possible.

An operation of acquiring the inverse of a spatial covariance matrix requires the largest computation amount. In a case in which the dimension of the spatial covariance matrix is L×L, O (L3) calculation is required to acquire the inverse matrix thereof. As the solution therefor, the input data is transformed from element space to another space, and then the components that affect less the performance of the MV BF are removed in said another space, so that the dimension of the spatial covariance matrix is drastically reduced, resulting in a simple calculation of the inverse of the covariance matrix. Such a method may, for example, include Fourier transform-based BA BF, PCA MV BF, etc.

The beamforming processor 12 uses orthogonal polynomials as a basis matrix for the space transform. The orthogonal polynomials may, compared to the BA BF, PCA MV BF methods, etc., generate less or similar approximation error, which is caused by the dimension reduction when the dimension of the spatial covariance matrix is reduced equally.

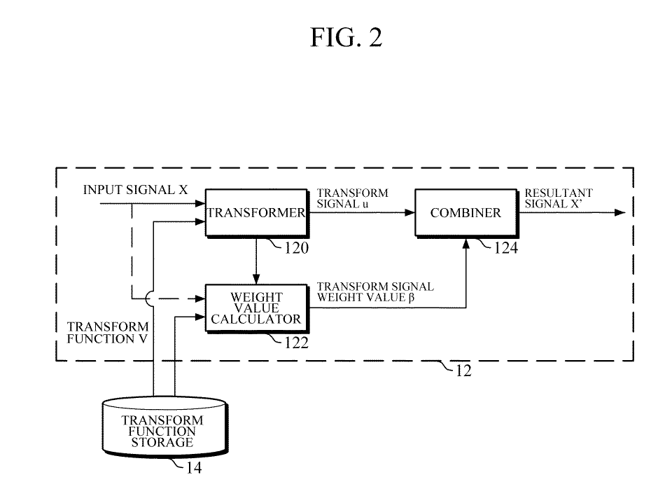

FIG. 2 is a detailed diagram illustrating an example of a beamforming processor according to an exemplary embodiment.

Referring to FIG. 2, a beamforming processor 12 includes a transformer 120, a weight value calculator 122, and a combiner 124.

The transformer 120 receives an input signal x from the outside, transforms the received input signal x by using a predetermined transform function V, and outputs the transform signal u, which is acquired after the input signal is transformed.

In one exemplary embodiment, the transformer 120 transforms the input signal x according to the transform function V that is set in advance by a user, or a system designer, etc. In another exemplary embodiment, the transformer 120 receives the transform function V for transforming the input signal x from transform function storage 14 that is built with at least one transform function V and transforms the input signal x by using the received transform function. The transform signal u generated by the transformer 120 is transmitted to the combiner 124.

The input signal x may be composed of a plurality of input signals that are input through a plurality of channels. That is, the input signal x may be a set of the input signals of the plurality of channels. Also, the transform signal u may be a set of the transform signals of the plurality of channels.

In a case in which the transform function V is given, the dimension of the transform signal u is smaller than that of the input signal x. Specifically, if the transform function is given as being a M×N matrix under the condition of M>N, and if the input signal is given as being a M×1 matrix (that is, if the input signal x is M-dimensional), the transform signal u, which is the calculation result thereof, is given as being a N×1 matrix so that the dimension of the transform signal u becomes smaller than the input signal x. As described, if the dimension is lowered, the computation amount thereof is relatively reduced so that the convenience and speed for the computation thereof may be improved.

The transform function V may be set in advance. In such a case, the transform function V may be acquired by additionally calculating in advance at least one transform function V based on various input signals x, which may be acquired through experiences or theories, to define at least one of the transform functions V, which are substituted or applied into the various input signals x. As described above, the transform function storage 14 may be built based on at least one of the transform functions V that is defined in advance.

The transformer 120 receives, from the transform function storage 14, a predetermined transform function V that has been acquired through such a process, and by using the received transform function V, generates a transform signal u. In such a case, the transform function V may be the combination of a plurality of basis vectors by, which has been selected by a user among the plurality of basis vectors that are stored in the transform function storage 14. That is, the transformer 120 may receive the plurality of basis vectors by in the process of receiving the transform function V, and use the transform function V generated by the combination of the received basis vectors by so as to transform the input signal x.

The generated transform signal u is transmitted to the combiner 124, and combined with a transform signal weight value β that is calculated by the weight value calculator 122, which will be specifically described later. As described above according to the exemplary embodiment, the combiner 120 may transmit, to the weight value calculator 122, which will be specifically described later, at least one of the input signal x and the transform function V that have been transmitted. The transformer 120 may transmit the transform signal u to the weight value calculator 122, which is, however, not illustrated in FIG. 2.

The weight value calculator 122 may calculate the transformed weight value β, which is the weight value added to the transform signal u that is output from the transformer 120. The weight value calculator 122 may calculate the transform signal weight value β by using at least one of the input signal x and the transform function V or both of them. In such a case, the weight value calculator 122 may receive the input signal x or the transform function V directly from a signal generator or the transform function storage 14, which generates signals, e.g., a transducer. Also, the weight value calculator 122 may receive, from the transformer 120, the above-mentioned input signal x or transform function V.

The weight value calculator 122 according to an exemplary embodiment calculates the transform signal weight value β based on the input signal x and the transform function V that is set in advance by a user, etc. or transmitted from an additional transform function storage 14, and then transmits the generated transform signal weight value β to the combiner 124.

The transform signal weight value β may be different according to the input signal x or the transform function V being used. The transform function V may be calculated in advance to be defined, and be used by being selected according to the input signal x so that the transform signal weight value β may be different mostly according to the input signal x.

The transform signal weight value β may be given in the form of a predetermined column vector, and if the transform function V is expressed in the form of an M×N matrix, the transform signal weight value β is given in the form of an N×1 matrix i.e., an N×1 column vector.

The combiner 124 generates a resultant signal x′ based on the transform signal u that has been generated and output from the transformer 120, and the transform signal weight value β that has been calculated from the weight value calculator 122. In such a case, the combiner 124 may generate the resultant signal x′ by combining the transform signal u and the transform signal weight value β, and for example, may generate a resultant signal x′ by calculating the weighted sum of the transform signal u and the transform signal weight value β. As a result, a beamforming apparatus may generate and output the resultant signal x′ which has been acquired after the beamforming is performed with regard to a predetermined input signal x.

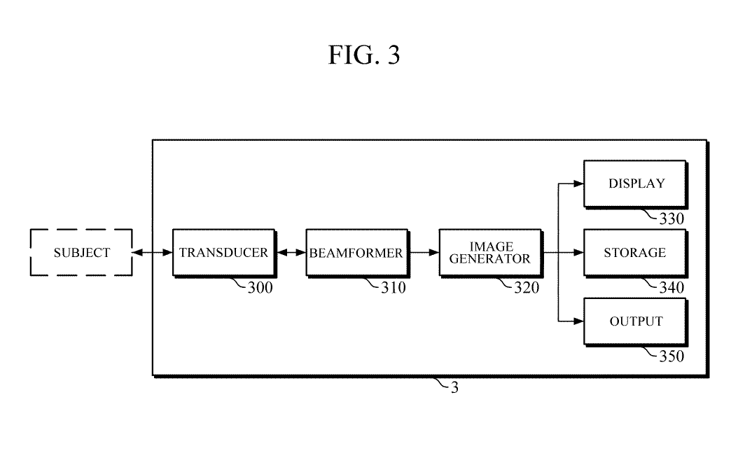

FIG. 3 is a diagram illustrating an example of an ultrasound imaging apparatus according to an exemplary embodiment.

Referring to FIG. 3, an ultrasound imaging apparatus 3 includes a transducer 300, a beamformer 310, an image generator 320, a display 330, storage 310, and an output 350.

The beamformer 310 is the exemplary embodiment of the beamforming apparatus 1 illustrated in FIGS. 1 and 2. Thus, what has been described above with relation to FIGS. 1 and 2 is also applicable to the ultrasound imaging apparatus illustrated in FIG. 3, so that the overlapped description is omitted here.

The ultrasound imaging apparatus 3 provides an image of a subject. For example, the ultrasound imaging apparatus 3 may display a diagnostic image showing a subject or output a signal that shows the diagnostic image regarding the subject to an external device, which displays the diagnostic image showing the subject. Here, the diagnostic image may be an ultrasonic image, to which exemplary embodiments thereof are, however, not limited.

The transducer 300 transmits and receives a signal to and from the subject. The transducer 300 transmits a transmission signal to the subject and receives an echo signal that has been reflected from the subject.

By using a plurality of basis vectors acquired from the beamforming coefficient of a pre-measured echo signal, the beamformer 310 calculates a weight value to be applied to the echo signal that has been reflected from the subject, applies the calculated weight value to the echo signal reflected from the subject, and combines the signals, to which the weight value is applied. Here, such a plurality of the basis vectors may be stored in the beamformer 310 or in the storage 340. Accordingly, the beamformer 310 may perform the beamforming by using at least a part of the plurality of the stored basis vectors. Here, the number of the basis vectors may be determined by a user. Likewise, by using the plurality of the basis vectors, the beamformer 310 calculates the weight value and then applies the calculated weight value so that the beamforming may be performed by using a reduced computation amount.

The image generator 320 generates an image by using the signals that have been output from the beamformer 310. The image generator 320 may include a digital signal processor (DSP) and a digital scan converter (DSC). According to an exemplary embodiment, the DSP performs a signal processing task of the signal that has been output from the beamformer 310; and the DSC generates an image by scanning and converting image data that is made using the signal, which is acquired after the signal processing task has been performed.

The display 330 displays the image that has been generated by the image generator 320. The display 330 may, for example, include output devices, such as a display panel, a mouse, an LCD screen, a monitor, which are equipped in the ultrasound imaging apparatus. Meanwhile, it will be understood by those skilled in the art that the ultrasound imaging apparatus 3 may not include the display 330, but may include the output 350 to output, to an external display, the image generated by the image generator 320.

The storage 340 stores the image that has been generated by the image generator 320, and the data that has been generated while an operation of the ultrasound imaging apparatus 3 is performed. The output 350 may transmit or receive data to or from an external device through a wired/wireless network, wired serial communication, etc. The external device may be another medical imaging system, a general-use computer system, a facsimile, etc. Also, it will be understood by those skilled in the art that the storage 340 and the output 350 may further include image decoding and searching functions to be integrated into the following form, such as a picture archiving communication system (PACS).

Since the computation amount being processed in performing the beamforming at the beamformer 310 is not large, the ultrasound imaging apparatus 3 may generate a real-time high-resolution image.

Hereinafter, various beamforming methods will be specifically described using the following equations.

A beamforming process of an ultrasonic diagnostic apparatus may be represented by Equation 1 below.

$$z [ n ] = {\sum }_{m = 0}^{M - 1} {w}_{m} [ n ] {x}_{m} [ n ] \qquad ( 1 )$$

In Equation 1, xm[n] indicates a reception signal of each channel to which a focusing delay is applied; m, a channel index; n, a time index; wm[n], weight values to be applied to the signal of each channel, which is also called as apodization; and z[n], an output of a beamforming apparatus.

MV BF may be used to acquire the variance of z[n], i.e., w that minimizes a power, while maintaining, as 1, the gain of the front signal in the desired direction, e.g., in the signal, to which the focusing delay is applied. Here, w[n]=[w0[n], w1[n], . . . , wm−1[n]]H. Thus, not giving any distortion to the signal in the desired direction, the MV BF may be used to minimize the degree, in which the signal in the undesired direction affects the output. Such an operation is represented by Equation 2 below.

$${min}_{w [ n ]} E [ {\vert x [ n ] \vert}^{2} ] = {min}_{w [ n ]} {w [ n ]}^{H} R [ n ] w [ n ] , \\ where \\ {w}^{H} [ N ] a = 1 \qquad ( 2 )$$

Here, E[·] indicates an expectation operator; w[n]H, the Hermitian transpose of w[n]; a, a steering vector; xm[n], a signal to which the focusing delay is applied, so that all the elements thereof are composed of 1. R[n] is a spatial covariance matrix as represented by Equation 3 below.

R[n]=E[x[n]x^{H}[n]] (3)

In Equation 3, x[n]=[x0[n], x1[n], . . . , xm−1[n]]T.

The solution of such an equation is represented by Equation 4 below.

$$w [ n ] = \frac{{R [ n ]}^{- 1} a}{{a}^{H} {R [ n ]}^{- 1} a} \qquad ( 4 )$$

In the actual situation, R[n] is required to be estimated, and so as to while being estimated, prevent the signal cancellation that is caused by the coherence of each of the channel signals, the spatial smoothing or the sub-aperture averaging is performed. Then, so as to improve statistical characteristics of the speckle pattern of a resultant image, the temporal averaging is performed. What is mentioned here is resented by Equation 5 below.

$$\begin{matrix}R [ n ] = \frac{1}{2 K + 1} \frac{1}{M - L + 1} {\sum }_{k = - K}^{K} {\sum }_{l = 0}^{M - L} {x}_{l} [ n - k ] {x}_{l}^{H} [ n - k ] , \\ where & ( 5 ) \\ {x}_{l} [ n ] = [ \begin{matrix}{x}_{l} [ n ] \\ {x}_{l + 1} [ n ] \\ ⋮ \\ {x}_{l + L - 1} [ n ]\end{matrix} ] & ( 6 )\end{matrix}$$

In Equation 5,

$$\frac{1}{M - L + 1} {\sum }_{l = 0}^{M - L}$$

corresponds to the spatial smoothing; and

$$\frac{1}{2 K + 1} {\sum }_{k = K}^{K} ,$$

the temporal averaging. In Equation 6, x[n] indicates a reception signal; and 1 from x1[n], a start index of x.

A diagonal loading method is mostly used to improve the robustness of the MV BF calculation, wherein the diagonal loading method is used to substitute \tilde{R}[n]+εI for \tilde{R}[n], where

ε=Δ·tr(\tilde{R}[n]). (7)

In Equation 7, tr( ) indicates a trace operator; and Δ, a constant which is called as a diagonal loading factor.

The following calculation is required so as to obtain an MV BF output from the MV weight values, which have been acquired through the spatial smoothing.

$$\tilde{z} [ n ] = \frac{1}{M - L + 1} {\sum }_{i = 0}^{M - L} {w [ n ]}^{H} {x}_{l} [ n ] \qquad ( 8 )$$

Here, w[n] is calculated from \tilde{R}[n].

Equation 8 is rearranged in the form of Equation 1, as follows.

$$\tilde{z} [ n ] = \frac{1}{M - L + 1} {\sum }_{l = 0}^{M - L} {\sum }_{k = 0}^{L - 1} ( {w}_{k} [ n ] {x}_{k - l} [ n ] ) = \frac{1}{M - L + 1} {\sum }_{m = 0}^{M - 1} [ ( {\sum }_{k = 0}^{L - 1} {w}_{k} [ n ] {r}_{m - k} ) {x}_{m} [ n ] ] \\ where \\ {r}_{k} = \{ \begin{matrix}1 & for 0 \leq k < M - L + 1 \\ 0 & otherwise\end{matrix} , \qquad ( 9 )$$

Here, an apodization function of standard MV BF, which corresponds to wm in Equation 1, is rk, which is, in other words, the convolution of a rectangle window with a length M−L+1 and w[n]H. Since it is well-known that the CW beam pattern on a focal plane is a Fourier transform pair of the apodization functions, the beam pattern of the apodization function of the standard MV BF is consequently shown as the multiplication of the Fourier transform pair of the rectangle window, i.e., a sinc function, and the Fourier transform pair of w[n]H acquired from the minimum variance. The sinc function is gradually decreased overall as being far away from the center, and is used to assume that a clutter that stays far away from a main lobe may be reduced to a certain degree only through the spatial smoothing.

FIGS. 4A and 4B are diagrams illustrating an example of high-frequency component being removed through only spatial smoothing according to an exemplary embodiment.

Specifically, FIG. 4A illustrates a point target image of DAS BF, which is apodized into a rectangle window; and FIG. 4B, a point target image of MV BF, in which the spatial smoothing is performed in a case in which w[n] is defined as a rectangle function.

It is assumed that L=M/4, and a plane wave of the front direction is transmitted. Referring to FIG. 4B, it may be confirmed that an X-type and long side lobe, observed in the DAS BF illustrated in FIG. 4A, is appropriately removed only through the spatial smoothing of the MV BF. It may be confirmed that although the dimensionality reduction is performed to, by using such properties, deal with only the properties of the side lobe that is near the front and remove other high-frequency properties in the MV BF that uses an orthogonal polynomials-based transform function, e.g., the Legendre polynomials, the properties of the standard MV BF may be very well maintained.

Hereinafter, an MV BF method to transform the MV BF into another space, instead of applying x into the original space of x, i.e., the element space, and perform the MV BF in the transformed space will be specifically described.

If it is assumed that a transform function V is an L×L full rank matrix, and the columns of V are orthonormal to each other, any weight value w in Equation 4 may be shown in the linear combination of the columns of V, as follows

w=Vβ (10),

where β indicates an L×1 column vector.

Then, the solution of the MV BF with regard to the given V is calculated as follows.

$$β = \frac{{R}_{1}^{- 1} {v}_{1}}{{v}_{1}^{H} {R}_{1}^{- 1} {v}_{1}} \qquad ( 11 )$$

Here, R1=VHRV=E[u·uH]; u=VHx; and v1=VHa. That is, Equation 11 indicates the MV BF solution of the space, in which x is transformed into VH.

In the actual situation, the spatial smoothing is required so as to estimate \tilde{R}₁, which is calculated through Equation 12 below.

$${\tilde{R}}_{1} = \frac{1}{M - L + 1} {\sum }_{l = 0}^{M - L} {u}_{l} {u}_{l}^{H} \qquad ( 12 )$$

\tilde{R}₁ substitutes R1 of Equation 11. Here, ul=VHxl, where

$${u}_{l} = [ \begin{matrix}{u}_{l , 0} [ n ] \\ {u}_{l , 1} [ n ] \\ ⋮ \\ {u}_{l , L - 1} [ n ]\end{matrix} ] . \qquad ( 13 )$$

The following equation is an output of a beamforming apparatus in consideration of the spatial smoothing.

$$\tilde{z} = \frac{1}{M - L + 1} {\sum }_{l = 0}^{M - L} {β}^{H} {u}_{l} \qquad ( 14 )$$

Meanwhile, the MV BF methods in the transformed space may drastically reduce the computation amount, which is required to calculate the inverse matrix of \tilde{R}, by particularly using only some essential components in the transformed space. Since some of the first columns that composes V represent the essential components in terms of performing the MV BF calculation, an input signal x is transformed by using only such columns so that the dimension of \tilde{R} may be reduced. Thus, the computation amount shown when the inverse matrix thereof is calculated may be greatly reduced.

If \hat{V} indicates a subspace composed of the first columns of V, the following equation may be acquired.

\hat{V}=[v₀, v₁, . . . , v_{Q-1}] (15),

where Q≦L. Then, a weight value calculated by using such \hat{V} is as follows.

$$\hat{w} = \hat{V} \hat{β} \\ where \\ \hat{β} = \frac{{\hat{R}}_{1}^{- 1} {\hat{v}}_{1}}{{\hat{v}}_{1}^{H} {\hat{R}}_{1}^{- 1} {\hat{v}}_{1}} , \\ {\hat{R}}_{1} = E [ \hat{u} \cdot {\hat{u}}^{H} ] , \\ \hat{u} = {\hat{V}}^{H} {x}_{i} , \\ and \\ {\hat{v}}_{1} = {\hat{V}}^{H} a . \qquad ( 16 ) ,$$

In the actual situation, in a case in which \hat{R}₁ is estimated using spatial smoothing, \hat{R}₁ is represented as follows.

$$\begin{matrix}{\hat{R}}_{1} = \frac{1}{M - L + 1} {\sum }_{l = 0}^{M - L} {\hat{u}}_{l} {\hat{u}}_{l}^{H} , \\ where & ( 17 ) \\ {\hat{u}}_{l} = [ \begin{matrix}{u}_{l , 0} [ n ] \\ {u}_{l , 1} [ n ] \\ ⋮ \\ {u}_{l , Q - 1} [ n ]\end{matrix} ] & ( 18 )\end{matrix}$$

Hereinafter, transform functions in MV BF will be described by using the following equations.

A known transform function, which is used to reduce the inverse matrix calculation of the MV BF, is a Fourier transform matrix for the BA BF (which is also called as Butler matrix), a matrix that is acquired by PCA for PCA MV BF, etc.

Bm,n, which is the component of m-th row and n-th column of the Fourier transform matrix B∈CL,L, is represented by the following equation.

$${B}_{m , n} = \frac{1}{\sqrt{L}} {e}^{- j2π mn / L} \qquad ( 19 )$$

Such a Fourier transform matrix transforms element space into transformed space. Some of the first columns among them indicate low-frequency components, which correspond to a focal point direction and a beam component that is near the focal point. If it is assumed that interference mostly occurs near the front direction, the dimension may be efficiently reduced by using only such columns. Furthermore, the spatial smoothing has an effect for reducing the interference that exists far away from the front, as described above.

In PCA MV BF, which is another approach, the transform function V is acquired by applying the PCA to a set of a lot of MV weight values, which are calculated by using the standard MV BF in the environment that is similar to an actual image processing environment. Thus, the columns of such V are composed of main components, and may fully reduce the dimension by using only some of the first columns. Such columns also represent the front direction and the beam component that is near the front.

Hereinafter, the transform function composed of the Legendre polynomials according to exemplary embodiments will be described with reference to the following equations.

A beamforming apparatus according to an exemplary embodiment uses an LP MV BF method, which uses the matrix composed of the Legendre polynomials as a transform function to transform the signal of the element space to the transformed space. Hereinafter, compared to a case in which another transform function is used, the MV BF performance in a case of using the Legendre polynomials will be specifically described.

Some of the first columns of the transform function composed of the Legendre polynomials may fully represent the low-frequency components likewise the Fourier transform function. Each of the Legendre polynomials that have been acquired after a Gram-Schmidt orthonormalization process is applied to a series of polynomials {1,n,n2, . . . , nL−1} may be used as the column of V. In other words, V=P, P=[P0, P1, . . . , PL−1], where Pk indicates a k-th column; Pk=[P0k, P1k, . . . , P(L−1)k]T; and

$${p}_{mk} = {\sum }_{n = 0}^{k} {m}^{n} {c}_{nk} .$$

cₙₖ is determined by the Gram-Schmidt orthonormalization process.

For example, P2 is represented by the following equation below.

$${p}_{2} = [ \begin{matrix}{p}_{02} \\ {p}_{12} \\ {p}_{22} \\ ⋮ \\ {p}_{( L - 1 ) 2}\end{matrix} ] = [ \begin{matrix}{c}_{02} \\ {c}_{22} + {c}_{12} + {c}_{02} \\ 4 {c}_{22} + 2 {c}_{12} + {c}_{02} \\ ⋮ \\ {( L - 1 )}^{2} {c}_{22} + ( L - 1 ) {c}_{12} + {c}_{02}\end{matrix} ] \qquad ( 20 )$$

The columns of P represent the components from low-frequency to high-frequency in order.

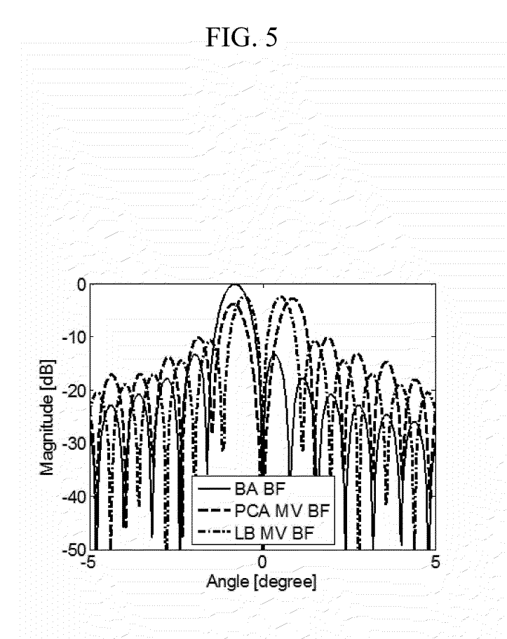

FIG. 5 is a diagram illustrating an example of comparing the continuous wave (CW) beam patterns of a transform function P based on Legendre Polynomials and the transform function of Fourier transform functions B and principal component analysis-based minimum variance beamforming (‘PCA MV BF’) method, according to an exemplary embodiment.

More specifically, FIG. 5 illustrates the comparison of the second column of P, i.e., CW beam patterns of P1, and CW beam patterns of the second columns of the transform function \hat{V} of the transform function B and the PCA MV BF.

In Equation 16 (i.e., ŵ=\hat{V}\hat{β}), since ŵ calculates a weighted sum of the first columns by using \hat{β}, the CW beam pattern of ŵ also calculate a weighted sum of the CW beam pattern of the columns of \hat{V} by using \hat{β}. It may be confirmed that the CW beam pattern from P has a quite similar pattern to the CW beam pattern of the transform function of the PCA MV BF.

However, P1 has the much lower frequency component than the second column b1 of B, and different from b1, the absolute value of the beam pattern thereof is symmetrical to 0. Such characteristics work as the advantages in that compared to the case in which B is used, the case in which P is used more effectively prevents the interference that exists close to the front even in the case of Q=2 so that a point target image may be sharper and symmetrical, as well.

Meanwhile, the direction of a null point in a CW beam pattern of one column of B is also the same as the null point direction even in the CW beam pattern of another column, but in the case of P, the direction thereof may be not the same, as shown in FIG. 2. For example, in the case of Q=2, if the interference is received from the first null point direction of the CW beam pattern of p1, such interference may not be removed even though any \hat{β} is used. That is because P1 does not have any influence with regard to that direction. Such a point may be almost the same even when the transform function of PCA MV BF is used, and may be the serious disadvantage when the CW is used. However, since various frequencies are mixed in an actual ultrasonic diagnostic device that uses wide-band signals, such a null point is not clearly shown in the beam pattern, which is not a big problem. However, rather with respect to the same dimension reduction, the LP MV BF's performance of decomposing the neighboring point targets is similar to or more excellent than other two methods; and with respect to the approximation error caused by the dimension reduction, the LP MV BF is almost always fewer than BA BF, and in most cases, does not have no difference from PCA MV BF.

One of the advantages of LP MV BF is that the transform function thereof is composed of only the real numbers. In principle, the BA BF or the PCA MV BF is required to be composed of complex numbers. Thus, the transform calculation becomes simple.

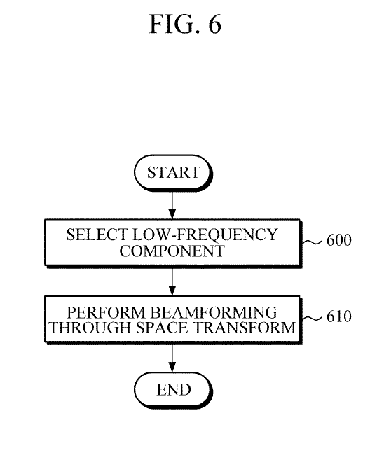

FIG. 6 is a flowchart illustrating an example of a beamforming method according to an exemplary embodiment.

Referring to FIG. 6, a beamforming apparatus according to an exemplary embodiment transforms an input signal of element space transformed space, which is another space, by using a transform function and generates and outputs a beam signal through signal processing in 610.

The beamforming apparatus uses a transform function that is composed of orthogonal polynomials when transforming the space. The orthogonal polynomials are a series of polynomials that meet the orthogonal relation. The orthogonal polynomials may be, for example, any one of the Hermite polynomials, the Laguerre polynomials, the Jacobi polynomials, the Gegenbauer polynomials, the Chebyshev polynomials, or the Legendre polynomials.

The beamforming apparatus uses, as a transform function, an orthonormal matrix composed of the Legendre polynomials when transforming the space. Specifically, the beamforming apparatus may use the Legendre polynomials as a transform function in a MV BF method. In such a case, while the computation amount of the MV BF is drastically reduced, its performance may be maintained.

The beamforming apparatus filters and acquires the low-frequency components in the element space in 610, which corresponds to a side lobe that is close to the front of a beam pattern by using a low-pass filter for beamforming. Here, the beamforming apparatus selects some of the first columns, which represent the low-frequency components, among the columns that compose the transform function. The reason why the low-pass filtering is performable is due to the fact that the high-frequency components, which exist far away from the front and in which the beam pattern is formed, are mostly removed through the spatial smoothing.

In a case in which the beamforming apparatus performs beamforming in transformed space by using a MV BF method, the beamforming apparatus may drastically reduce the computation amount, which is required for calculating the inverse of a spatial covariance matrix by using only some essential components in the transformed space. For example, the dimension of the spatial covariance matrix is reduced by selecting only some of the first columns among the transform functions and transforming the input signal. Some of the first columns among the transform functions represent essential components in the MV BF calculation.

In a case in which the beamforming apparatus performs an orthogonal polynomials-based MV BF method, only some of the first columns of the transform function composed of the orthogonal polynomials are used. Since such columns of the transform function represent the components from low-frequency to high-frequency in order, some of the first columns correspond to the low-frequency components the same as the Fourier-transform function.

An X-type and long side lobe, which is observed in a DAS BF method, may be removed through the spatial smoothing of the MV BF. Although the dimensionality reduction is performed to, by using such properties, deal with only the properties of the side lobe that is near the front and remove other high-frequency properties in the MV BF that uses the orthogonal polynomials-based transform function, the properties of the MV BF may be very well maintained. It will be confirmed that the high-frequency properties are removed only through the spatial smoothing, with reference to FIGS. 4A and 4B that will be described later.

As described above, exemplary embodiments may be all applied to various array signal processing fields, such as radar, the sonar, the non-destructive inspection, etc., as well as an ultrasound imaging apparatus.

In an exemplary embodiment, in the process of performing the beamforming of an input signal by using a beamforming apparatus, an ultrasound imaging apparatus, and a beamforming method, and acquiring a resultant signal, a required computation amount may be reduced. Thus, all types of devices that perform the beamforming, e.g., an ultrasound imaging apparatus may reduce the resources required for the beamforming. Particularly, the input signal is transformed into new space by using a transform function that is composed of orthogonal polynomials so that a complexity in calculating the inverse of the covariance matrix in a MV BF method may be drastically reduced.

In addition, the beamforming speed with regard to the input signal may be fast so that the duration of the beamforming process may be reduced. Moreover, a beamforming apparatus may also acquire effects of solving all types of problems, such as the delayed output of an ultrasound image, overload or overheat of the device. Furthermore, the following effects may be acquired: the reduction of the power consumed in the beamforming apparatus, which is caused by the reduction of the resource use amount in the beamforming apparatus; and the cost reduction, which is caused by the use of a computation device with a low specification.

Exemplary embodiments may be all applied to various array signal processing fields, such as radar, the sonar, the non-destructive inspection, etc., including an ultrasound imaging apparatus.

A number of examples have been described above. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A beamforming apparatus, comprising:
a filter configured to, among components of a transform function, remove high-frequency components and select low-frequency components; and
a beamforming processor configured to transform an input signal to another space by using the transform function composed of the selected low-frequency components, and generate a beam signal through signal processing in the transformed space.

2. The beamforming apparatus of claim 1, wherein the transform function is composed of orthogonal polynomials.

3. The beamforming apparatus of claim 2, wherein the orthogonal polynomials are one of Hermite polynomials, Laguerre polynomials, Jacobi polynomials, Gegenbauer polynomials, Chebyshev polynomials, the Legendre polynomials.

4. The beamforming apparatus of claim 3, wherein a transform function V is Legendre polynomials P, where P=[P0, P1, . . . , PL−1]T,
$${p}_{mk} = {\sum }_{n = 0}^{k} {m}^{n} {c}_{nk} ,$$
Pk is a k-th column of P, and cₙₖ is determined by a Gram-Schmidt orthonormalization process.

5. The beamforming apparatus of claim 2, wherein the beamforming processor is configured to perform beamforming by using a minimum variance that is based on the orthogonal polynomials in the transformed space.

6. The beamforming apparatus of claim 1, wherein the beamforming processor comprises:
a transformer configured to generate a transform signal with regard to an input signal by using the transform function;
a weight value calculator configured to calculate a transform signal weight value, which is a weight value with regard to the transform signal; and
a combiner configured to generate a beam signal by using the transform signal and the transform signal weight value.

7. The beamforming apparatus of claim 6, wherein the weight value calculator is configured to calculate the weight value from a spatial covariance matrix, which is generated through spatial smoothing for generating the spatial covariance matrix from the transform signal.

8. An ultrasound imaging apparatus, comprising:
a transducer configured to irradiate ultrasonic waves to a subject, receive a signal of the ultrasonic waves reflected from the subject, transform the received ultrasonic waves, and output a plurality of the ultrasonic signals;
a beamformer configured to transform, to another space, the signal of the ultrasonic waves, which has been input through the transducer, by using a transform function, generate a beam signal through signal processing in the transformed space, among components of the transform function, remove high-frequency components, and select and process low-frequency components; and
an image generator configured to generate an image by using a beam signal, which has been generated by the beamformer.

9. The ultrasound imaging apparatus of claim 8, wherein the transform function is composed of orthogonal polynomials.

10. The ultrasound imaging apparatus of claim 9, wherein the orthogonal polynomials are one of Hermite polynomials, Laguerre polynomials, Jacobi polynomials, Gegenbauer polynomials, Chebyshev polynomials, the Legendre polynomials.

11. The ultrasound imaging apparatus of claim 10, wherein a transform function V are Legendre polynomials P, where P=[P0, P1, . . . , PL−1]T,
$${p}_{mk} = {\sum }_{n = 0}^{k} {m}^{n} {c}_{nk} ,$$
Pk is a k-th column of P, and cₙₖ is determined by a Gram-Schmidt orthonormalization process.

12. The ultrasound imaging apparatus of claim 9, wherein the beamformer is configured to perform beamforming by using a minimum variance that is based on the orthogonal polynomials in the transformed space.

13. A beamforming method, comprising:
removing high-frequency components and selecting low-frequency components among components of a transform function; and
transforming an input signal to another space by using the transform function composed of the selected low-frequency components, and generating a beam signal through signal processing in the transformed space.

14. The beamforming method of claim 13, wherein the transform function is composed of orthogonal polynomials.

15. The beamforming method of claim 14, wherein the orthogonal polynomials are one of Hermite polynomials, Laguerre polynomials, Jacobi polynomials, Gegenbauer polynomials, Chebyshev polynomials, the Legendre polynomials.

16. The beamforming method of claim 15, wherein a transform function V are Legendre polynomials P, where P=[P0, P1, . . . , PL−1]T,
$${p}_{mk} = {\sum }_{n = 0}^{k} {m}^{n} {c}_{nk} ,$$
Pk is a k-th column of P, and cₙₖ is determined by a Gram-Schmidt orthonormalization process.
